(19) United States
(12) Patent Application Publication
(10) Pub. No.: US 2015/0290333 A1
(43) Pub. Date: Oct. 15, 2015

(54) PROCESS AND MATERIALS FOR MEDICAL APPLICATIONS

(71) Applicant: NATIONAL UNIVERSITY OF SINGAPORE, Singapore (SG)

(72) Inventors: Michael Raghunath, Singapore (SG); Sebastian Beyer, Singapore (SG); Rafi Rashid, Singapore (SG); Anna Maria Blocki, Singapore (SG); Dieter Trau, Singapore (SG); Thorsten Wohland, Singapore (SG)

(73) Assignee: National University of Singapore, Singapore (SG)

(21) Appl. No.: 14/443,190

(22) PCT Filed: Oct. 30, 2013
(86) PCT No.: PCT/SG2013/000465

§ 371 Date: May 15, 2015

(30) Foreign Application Priority Data
Nov. 16, 2012 (GB) 1220675.1

(51) Int. Cl. A61K 47/48; A61K 48/00; A61K 39/44; A61K 31/7052; A61K 31/203; A61K 31/337

(52) U.S. Cl. CPC A61K 47/48092; A61K 47/4823; A61K 47/48884; A61K 31/203; A61K 31/337; A61K 39/44; A61K 31/7052; A61K 48/00

(57) ABSTRACT

This disclosure relates to a fully or partially biodegradable carrier for the delivery of biologically active agents which are associated, either directly or indirectly, with the carrier via a biodegradable linking agent and the use of the carrier in the delivery of bioactive molecules for therapy and imaging, in particular the delivery of agents to mitochondria.

### FIELD OF THE INVENTION

This disclosure relates to a fully or partially biodegradable carrier for the delivery of biologically active agents which are associated or cross-linked to the carrier, either directly or indirectly, via a cleavable and preferably biodegradable linking agent; a process for the manufacture of the carrier and agent; pharmaceutical compositions comprising the carrier and agent, wherein the carrier comprises materials that are adapted to be metabolised to none toxic degradation products. Advantageously, in some embodiments, the carrier can accumulate intra-cellularly in organelles, such as mitochondria.

### BACKGROUND TO THE INVENTION

The application of nanoparticles in biology and medicine has rapidly grown in recent years due to their advantageous physical and chemical properties. Nanoparticles can be found composed of a variety of inorganic or organic materials, and are used in various biomedical applications such as tissue engineering, biomarkers, labelling and tracking agents, vectors for gene therapy, hyperthermia treatments and magnetic resonance imaging (MRI), contrast agents and drug delivery.

For the purpose of drug delivery, nanoparticles are defined as biocompatible submicron sized particles (<1 μm) in which the desired drug is dissolved or covalently attached. Nanoparticles have to fulfil a wide range of often conflicting technical characteristics to be useful in biomedical applications. It is essential for nanoparticles to be highly stable to allow targeted drug delivery and sustained release. Nanoparticles are desired to have amphiphilic properties permitting the transport of both hydrophilic and hydrophobic compounds and offer suitability for chemical modification, which limits often the choices of materials. Additionally, nanoparticles have to be tailored to fit various routes of administration as oral administration or inhalation. Another important aspect is that nanoparticles are composed of biocompatible, biodegradable material such as synthetic or natural polymers or lipids to minimise the risk of rejection and avoid degradation to toxic components.

Organic biodegradable polymers such as polyhydroxybuterate (PHB), poly lactic acid (PLA), poly caprolactam (PCL), poly amino acids, poly amides, poly glycidols and others are currently considered as suitable materials for the development of nanoparticles for drug delivery. However, the biodegradability of those compounds is debatable as the cleaved monomers are substances which can't be further metabolised and therefore are often associated with inflammatory responses. In addition these polymers suffer from a further disadvantage in so far as their degradation can be delayed in vivo.

Polycarbohydrates [for example dextran, cellulose, pullulan]derived from natural sources offer a suitable alternative as material for nano particulate formulations. However, formulations comprising such polycarbohydrates are often characterised by a reduced or limited ability to degrade under physiological conditions and their prolonged presence in physiological systems is associated with the formation of inclusions, oxidative stress and possible inflammation. Other suitable bioactive biomolecules require stabilisation with polyethyleneglycol (PEG). PEG is used in a variety of pharmaceutical products as laxative, tablet binders or lubricants. In protein medication PEG is used as a stabiliser which results in slow clearance and reduced toxicity. However, pegylation of bioactive compounds reduces the affinity of the modified agent for its target necessitating administration of increased medicinally doses which potentially results in an increased side effect profile. PEG has been used in a variety of nano-based technologies. For example US application US2011/0244048 discloses the use of PEG for the construction of dextran-based nanoparticles to allow the crosslinking of a targeting ligand, and cellulose-based nanoparticles for drug delivery covalently linked to PEG are disclosed in patent application US2012/0219508.

The present disclosure relates to a fabrication process forming nano-sized materials composed of carbohydrate based building units, bioactive components and, agents that act as a linkage, either covalent or non-covalent, between the bioactive component and the carrier. Nanoparticles built in this way provide an entirely new class of materials allowing drug delivery in a completely biodegradable carrier. The carbohydrate based building units allow non-covalent and covalent immobilisation of both hydrophilic and hydrophobic target molecules.

### STATEMENTS OF INVENTION

According to an aspect of the invention there is provided an oligomeric carrier complex comprising:
i) a carbohydrate based polymer;
ii) one or more biologically active molecules;
iii) a linkage agent that either directly or indirectly links or associates the carbohydrate polymer with the biologically active molecule[s], wherein the polymer and linkage agent are biodegradable and the polymer and/or linkage agent is adapted to be metabolized by an organism or cell after administration; and optionally
iv) a targeting agent to target the complex to a cell or organ.

In a preferred embodiment of the invention said complex is a nanoparticle.

In a preferred embodiment of the invention said complex has a diameter 1-1000 nm.

In an alternative preferred embodiment of the invention said complex has a diameter of 1-300 nm; preferably 5-100 nm.

In a preferred embodiment of the invention said carbohydrate carrier comprises a sugar capable of being metabolized by a cell.

In a preferred embodiment of the invention said carbohydrate based polymer comprises or consists essentially of one or more monomeric sugars.

In a preferred embodiment of the invention said monomer sugar is selected from the group: glucose, galactose or fructose. Preferably said monomeric sugar is glucose.

In an alternative preferred embodiment of the invention said carbohydrate based polymer consists essentially of one or more dimeric sugars.

In a preferred embodiment of the invention said dimeric sugar is selected from the group consisting of: sucrose, lactose or maltose. Preferably said dimeric sugar is sucrose, for example polysucrose.

In a preferred embodiment of the invention said carbohydrate based polymer comprises one or more different monomeric or dimeric sugars.

In an alternative preferred embodiment of the invention said carbohydrate based polymer comprises one or more modified sugars.

In a preferred embodiment of the invention said biologically active molecule is a therapeutic agent.

In a preferred embodiment of the invention said therapeutic agent is a small organic molecule.

In a preferred embodiment of the invention said organic molecule is a chemotherapeutic agent.

In an alternative preferred embodiment of the invention said small organic molecule is an antibiotic.

In a further alternative embodiment of the invention said small organic molecule is an antiviral agent.

In an alternative preferred embodiment of the invention said therapeutic agent is proteinaceous.

In a preferred embodiment of the invention said proteinaceous therapeutic agent is a therapeutic antibody, or an active binding fragment thereof.

In a preferred embodiment of the invention said antibody is a monoclonal antibody.

In a preferred embodiment of the invention said antibody is a chimeric antibody.

In an alternative preferred embodiment of the invention said antibody is a humanized or human antibody.

In an alternative preferred embodiment of the invention said active binding fragment is selected from the group: Fab, Fab₂, F(ab′)₂, Fv, Fc, Fd, single chain antibody fragment.

In a preferred embodiment of the invention said fragment is a single chain antibody fragment.

In an alternative preferred embodiment of the invention said proteinaceous agent is non-antibody pharmaceutical peptide or protein.

In a further alternative preferred embodiment of the invention said therapeutic agent is a nucleic acid.

In a preferred embodiment of the invention said nucleic acid agent comprises an antisense RNA or an antisense oligonucleotide.

In a preferred embodiment of the invention said nucleic acid agent is a small interfering RNA [siRNA].

In a preferred embodiment of the invention said antisense oligonucleotide or siRNA includes modified nucleotides.

In an alternative preferred embodiment of the invention said nucleic acid is a gene therapy vector adapted for expression.

In a preferred embodiment of the invention said gene therapy vector is viral based.

In a preferred embodiment of the invention said biologically active agent is associated with a second carrier moiety which is crosslinked or associated with the carrier according to the invention.

In a preferred embodiment of the invention said second carrier is biodegradable and preferably adapted to be metabolized.

In a preferred embodiment of the invention said second carrier is a dextrin, preferably a cyclic dextrin.

In a preferred embodiment of the invention said targeting agent is an antibody or antibody fragment thereof.

In a preferred embodiment of the invention the antibody or antibody fragment functions as both the biologically active agent and targeting agent.

In an alternative preferred embodiment of the invention said targeting agent is a ligand for a receptor expressed by a target cell or organ and targeting of the oligomeric carrier complex is via ligand:receptor binding.

In an alternative preferred embodiment of the invention said biologically active agent is an imaging agent.

In a preferred embodiment of the invention said linking agent is cleavable and biodegradable.

In a preferred embodiment of the invention said linking agent forms a covalent linkage between the carbohydrate polymer and the biologically active agent[s].

In an alternative preferred embodiment of the invention said linking agent forms a non-covalent linkage between the carbohydrate polymer and the biologically active agent[s].

Preferably said linking agent is biodegradable and is adapted to be metabolized by an organism or cell after administration.

According to a further aspect of the invention there is provided a pharmaceutical composition comprising an effective amount of an oligomeric carrier complex according to the invention and including a physiologically acceptable excipient.

According to a further aspect of the invention there is provided an oligomeric carrier complex according to the invention for use as a medicine.

According to a further aspect of the invention there is provided an oligomeric carrier complex according to the invention for use in the treatment of cancer.

According to a further aspect of the invention there is provided an oligomeric carrier complex crosslinked or associated with a nucleic acid based vector for use in the transfection of eukaryotic cells.

In a preferred embodiment of the invention said eukaryotic cell is a mammalian cell; preferable a human cell.

According to a further aspect of the invention there is provided an oligomeric carrier complex according to the invention for use in the treatment of acute or chronic wounds.

According to a further aspect of the invention there is provided an ex vivo method for the administration of an oligomeric carrier complex comprising the steps:
i) forming a preparation comprising an isolated cell sample obtained from a subject and an oligomeric carrier complex according to the invention;
ii) incubating the cell preparation under conditions that allow the uptake of the oligomeric carrier complex into one or more cell types contained in the sample; and optionally
iii) re-administering the cell preparation to said subject.

In a preferred method of the invention said sample is a blood sample.

In a preferred method of the invention said cell type is a blood immune cell.

Preferably said blood cell is selected from the group consisting of peripheral blood mononuclear cells [PBMCs].

In a T-lymphocytes, [either or both CD8⁺ T lymphocytes or CD4⁺ T lymphocytes] B lymphocytes, Dendritic Cells, T Regulatory Cells, innate lymphoid cells or Natural Killer Cells [NK cells].

In an alternative preferred embodiment of the invention said cell is a stem cell, preferably a mesenchymal stem cell.

According to a further aspect of the invention there is provided a process for the manufacture of a biodregadable oligomeric carrier complex comprising the steps:
i) forming a reaction mixture comprising a carbohydrate based carrier, a biologically active agent and optionally a cross-linking agent;
ii) incubating the reaction mixture under reaction conditions that cross-link the carbohydrate based carrier and the biologically active agent to form an oligomeric carrier complex; and optionally
iii) purifying the oligomeric carrier complex from the reaction mixture.

In a preferred method of the invention said reaction mixture includes a cleavable cross-linking agent.

In a preferred method of the invention said cross-linking agent is an organic cross-linking agent and is cleavable and preferably biodegradable, for example an amino acid based or modified amino acid based cross-linking agent.

In an alternative preferred method of the invention said cross-linking agent is the biologically active agent.

According to a further aspect of the invention there is provided an oligomeric carrier complex for use in the delivery of one or more agents to mitochondria.

In a preferred embodiment of the invention said agent is a therapeutic agent.

In a preferred embodiment of the invention said agent is effective in the treatment of diseases or conditions that result from mitochondrial dysfunction.

In a preferred embodiment of the invention said disease or condition is selected from the group consisting of: neurodegenerative diseases, cancer, cardiovascular diseases, diabetes or related metabolic diseases.

In a preferred embodiment of the invention said agent is a photo-sensitizing agent.

In an alternative preferred embodiment of the invention said agent is a nucleic acid based agent, for example an antisense nucleic acid directed to a mitochondrial gene or a nucleic acid comprising a mitochondrial DNA construct capable of recombination with mitochondrial DNA.

In an alternative preferred embodiment of the invention said agent as an imaging agent.

In a preferred embodiment of the invention said imaging agent comprises a fluorophore.

In a preferred embodiment of the invention said agent is both a therapeutic agent and an imaging agent.

### EMBODIMENTS OF THE INVENTION

### Carbohydrate Based Polymer

The invention utilises polymers comprising sugars that are biodegradable and uniquely also capable of being metabolized by cells/organs. This advantageously provides a particulate complex, preferably a nano-particle which is efficiently removed from the circulation once the biologically active agent is delivered and is also utilized by the organism either as an energy source or in intermediate metabolism. The polymer can be manufactured using any sugar or modified sugar that is metabolized to non-toxic waste products. The polymer comprises monomeric, dimeric and oligomeric sugar units and mixtures thereof with the objective to provide a fully biodegradable carrier.

### Small Organic Molecules

A general definition of “chemotherapeutic agent” is an agent that typically is a small chemical compound that preferably kills cells in particular diseased cells or is at least cytostatic. Agents can be divided with respect to their structure or mode of action. For example, chemotherapeutic agents include alkylating agents, anti-metabolites, anthracyclines, alkaloids, plant terpenoids and toposisomerase inhibitors. Chemotherapeutic agents typically produce their effects on cell division or DNA synthesis. Examples of alkylating agents are is cisplatin, carboplatin or oxaliplatin. Examples of anti-metabolites include purine or pyrimidine analogues. Purine analogues are known in the art. For example thioguanine is used to treat acute leukaemia. Fludarabine inhibits the function of DNA polymerases, DNA primases and DNA ligases and is specific for cell-cycle S-phase. Pentostatin and cladribine are adenosine analogues and are effective against hairy cell leukaemias. A further example is mecrcaptopurine which is an adenine analogue. Pyrimidine analogues are similarly known in the art. For example, 5-fluorouracil (5-FU), floxuridine and cytosine arabinoside. 5-FU has been used for many years in the treatment of breast, colorectal cancer, pancreatic and other cancers. 5-FU can also been formed from the pro-drug capecitabine which is converted to 5-FU in the tumour. Leucovorin, also known as folinic acid, is administered as an adjuvant in cancer chemotherapy and which enhances the inhibitory effects of 5-FU on thymidylate synthase. Alkylating agents are also known in the art and include vinca alkaloids, for example vincristine or vinblastine. Terpenoids have been used for many years and include the taxanes, for example, palitaxel.

Antibiotics and antiviral agents are effective in the treatment of microbial, for example bacterial and parasitic pathogens and pathogenic viruses. The carrier according to the invention is particularly well suited to the treatment of intracellular microbial pathogens. For example species of the genus Mycobacterium, Brucella, Francisella, Legionella and Listeria can exist in an intracellular form. Other bacterial species either are intracellular or are obligate intracellular species, for example species of the genera Chlamydia, Rickettsia, Salmonella and Yersinia. Viruses are of course obligate intracellular parasites. Parasitic microbial intracellular pathogens include species of the genera Plasmodia, Toxoplasma, Leishmania and the trypanosomatid species Trypanosoma cruzi. Examples of classes of antibiotics effective in the control of bacterial pathogens include, by example only, penicillins, cephalosporins, rifamycins, sulphonomides, macrolides and tetracyclines. Also included within the scope of the invention are antibacterial peptides such as dermicidins, cecropins and defensins. Antiviral agents include anti-retroviral drugs such as zidovudine, lamivudine, efavrenz and abacavir; and anti-viral drugs such as ganciclovir, aciclovir and oseltamivir. Anti-protozoan agents include lumefantrine, mefloquine, amodiaquine, sulfadoxine, chloroquine used in the treatment of malaria and also combination therapies that use these agents in combination with artemisinin. These are non-limiting examples of agents that can be used with the carrier according to then invention.

### Antibodies

Antibodies include polyclonal and monoclonal antibodies, prepared according to conventional methodology.

Chimeric antibodies are recombinant antibodies in which all of the V-regions of a mouse or rat antibody are combined with human antibody C-regions. Humanised antibodies are recombinant hybrid antibodies which fuse the complementarity determining regions from a rodent antibody V-region with the framework regions from the human antibody V-regions. The C-regions from the human antibody are also used. The complementarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

Various fragments of antibodies are known in the art. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof. A fragment of an antibody or immunoglobulin can also have bispecific function as described above.

### Pharmaceutical Proteins

Examples of pharmaceutical proteins include “cytokines”. Cytokines are involved in a number of diverse cellular functions. These include modulation of the immune system, regulation of energy metabolism and control of growth and development. Cytokines mediate their effects via receptors expressed at the cell surface on target cells. Examples of cytokines include the interleukins such as: IL1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 and 33.

Other examples include growth hormone, leptin, erythropoietin, prolactin, tumour necrosis factor [TNF] granulocyte colony stimulating factor (GCSF), granulocyte macrophage colony stimulating factor (GMCSF), ciliary neurotrophic factor (CNTF), cardiotrophin-1 (CT-1), leukemia inhibitory factor (LIF) and oncostatin M (OSM), interferon α, interferon β, interferon ε, interferon κ and ω interferon.

Examples of pharmaceutically active peptides include GLP-1, anti-diuretic hormone; oxytocin; gonadotropin releasing hormone, corticotrophin releasing hormone; calcitonin, glucagon, amylin, A-type natriuretic hormone, B-type natriuretic hormone, ghrelin, neuropeptide Y, neuropeptide YY₃₋₃₆, growth hormone releasing hormone, somatostatin; or homologues or analogues thereof.

The term “chemokine” refers to a group of structurally related low-molecular weight factors secreted by cells having mitogenic, chemotactic or inflammatory activities. They are primarily cationic proteins of 70 to 100 amino acid residues that share four conserved cysteine residues. These proteins can be sorted into two groups based on the spacing of the two amino-terminal cysteines. In the first group, the two cysteines are separated by a single residue (C-x-C), while in the second group they are adjacent (C—C). Examples of member of the ‘C-x-C’ chemokines include but are not limited to platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF),and IP10. Examples of members of the ‘C—C’ group include but are not limited to monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1β (MIP-1-β), macrophage inflammatory protein 1-γ (MIP-1-γ), macrophage inflammatory protein 3α (MIP-3-α, macrophage inflammatory protein 3β (MIP-3-β), chemokine (ELC), macrophage inflammatory protein-4 (MIP-4), macrophage inflammatory protein 5 (MIP-5), LD78β, RANTES, SIS-epsilon (p500), thymus and activation-regulated chemokine (TARC), eotaxin, 1-309, human protein HCC-1/NCC-2, human protein HCC-3.

A number of growth factors have been identified which promote/activate endothelial cells to undergo angiogenesis. These include vascular endothelial growth factor (VEGF A); VEGF B, VEGF C, and VEGF D; transforming growth factor (TGFb); acidic and basic fibroblast growth factor (aFGF and bFGF); and platelet derived growth factor (PDGF). VEGF is an endothelial cell-specific growth factor which has a very specific site of action, namely the promotion of endothelial cell proliferation, migration and differentiation. VEGF is a complex comprising two identical 23 kD polypeptides. VEGF can exist as four distinct polypeptides of different molecular weight, each being derived from an alternatively spliced mRNA. bFGF is a growth factor that functions to stimulate the proliferation of fibroblasts and endothelial cells. bFGF is a single polypeptide chain with a molecular weight of 16.5 Kd. Several molecular forms of bFGF have been discovered which differ in the length at their amino terminal region. However the biological function of the various molecular forms appears to be the same.

Pro-drug activating polypeptides are also within the scope of the invention. The term pro-drug activating genes refers to nucleotide sequences, the expression of which, results in the production of proteins capable of converting a non-therapeutic compound into a therapeutic compound, which renders the cell susceptible to killing by external factors or causes a toxic condition in the cell. An example of a prodrug activating gene is the cytosine deaminase gene. Cytosine deaminase converts 5-fluorocytosine to 5 fluorouracil, a potent antitumour agent. The lysis of the tumour cell provides a localized burst of cytosine deaminase capable of converting 5FC to 5FU at the localized point of the tumour resulting in the killing of many surrounding tumour cells. Additionally, the thymidine kinase (TK) gene (see U.S. Pat. No. 5,631,236 and U.S. Pat. No. 5,601,818) in which the cells expressing the TK gene product become susceptible to selective killing by the administration of ganciclovir may be employed. Other examples of pro-drug activating enzymes are nitroreductase and cytochrome p450's (e.g. CYP1A2, CYP2E1 or CYP3A4).

### Inhibitory RNA

A technique to specifically ablate gene function which has broad acceptance is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

### Modified Nucleic Acid Molecules

The term “modified” as used herein describes a nucleic acid molecule in which;
i) at least two of its nucleotides are covalently linked via a synthetic internucleoside linkage (i.e., a linkage other than a phosphodiester linkage between the 5′ end of one nucleotide and the 3′ end of another nucleotide). Alternatively or preferably said linkage may be the 5′ end of one nucleotide linked to the 5′ end of another nucleotide or the 3′ end of one nucleotide with the 3′ end of another nucleotide; and/or
ii) a chemical group, such as cholesterol, not normally associated with nucleic acids has been covalently attached to the double stranded nucleic acid.
iii) Preferred synthetic internucleoside linkages are phosphorothioates, alkylphosphonates, phosphorodithioates, phosphate esters, alkylphosphonothioates, phosphoramidates, carbamates, phosphate triesters, acetamidates, peptides, and carboxymethyl esters.

The term “modified” also encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5 carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Modified double stranded nucleic acids also can include base analogs such as C-5 propyne modified bases (see Wagner et al., Nature Biotechnology 14:840-844, 1996).

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and thereby, inhibits the transcription of that gene and/or the translation of that mRNA. The antisense molecules are designed so as to interfere with transcription or translation of a target gene upon hybridization with the target gene. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. In order to be sufficiently selective and potent for inhibition, such antisense oligonucleotides should comprise at least 7 (Wagner et al., Nature Biotechnology 14:840-844, 1996) and more preferably, at least 15 consecutive bases which are complementary to the target. Most preferably, the antisense oligonucleotides comprise a complementary sequence of 20-30 bases.

### Gene Therapy

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, preferably selected from baculoviridiae, parvoviridiae, picornoviridiae, herpesveridiae, poxviridae, adenoviridiae, or picornnaviridiae. Chimeric vectors may also be employed which exploit advantageous elements of each of the parent vector properties (see e.g., Feng, et al. (1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443; Henderson, et al., U.S. Pat. No. 5,871,726 the entire teachings of which are herein incorporated by reference. It has been demonstrated that viruses which are attenuated for replication are also useful in gene therapy. For example the adenovirus dI1520 containing a specific deletion in the E1b55K gene (Barker and Berk (1987) Virology 156: 107) has been used with therapeutic effect in human beings. Such vectors are also described in McCormick U.S. Pat. No. 5,677,178 and U.S. Pat. No. 5,846,945.

Certain vectors exhibit a natural tropism for certain tissue types. For example, vectors derived from the genus herpesviridiae have been shown to have preferential infection of neuronal cells. Examples of recombinant modified herpesviridiae vectors are disclosed in U.S. Pat. No. 5,328,688. Cell type specificity or cell type targeting may also be achieved in vectors derived from viruses having characteristically broad infection by the modification of the viral envelope proteins. For example, cell targeting has been achieved with adenovirus vectors by selective modification of the viral genome knob and fibre coding sequences to achieve expression of modified knob and fibre domains having specific interaction with unique cell surface receptors. Other methods of cell specific targeting have been achieved by the conjugation of antibodies or antibody fragments to the envelope proteins (see, e.g. Michael, et al. (1993) J. Biol. Chem 268:6866-6869, Watkins, et al. (1997) Gene Therapy 4:1004-1012; Douglas, et al (1996) Nature Biotechnology 14: 1574-1578. Alternatively, particularly moieties may be conjugated to the viral surface to achieve targeting (see, e.g. Nilson, et al. (1996) Gene Therapy 3:280-286 (conjugation of EGF to retroviral proteins).

### Pharmaceutical Formulations

When administered the compositions of the present invention are administered in pharmaceutically acceptable preparations. Such preparations may routinely contain pharmaceutically acceptable concentrations of salt, buffering agents, preservatives, compatible carriers and supplementary therapeutic agents' [e.g. anti-cancer agents].

The compositions of the invention can be administered by any conventional route, including injection or by gradual infusion over time. The administration may, for example, intravenous, intraperitoneal, intramuscular, intracavity, subcutaneous, transdermal or trans-epithelial.

The compositions of the invention are administered in effective amounts. An “effective amount” is that amount of an agent that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The compositions used in the foregoing methods preferably are sterile and contain an effective amount of an agent according to the invention for producing the desired response in a unit of weight or volume suitable for administration to a patient. The doses of agent administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of between 1 nM-1 mM generally will be formulated. Preferably doses can range from 1 nM-500 nM, 5 nM-200 nM, and 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the compositions of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents' (e.g. those typically used in the treatment of the specific disease indication). When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions containing agents according to the invention may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The compositions may conveniently be presented in unit dosage form and may be prepared by any of the methods well-known in the art of pharmacy. All methods include the step of bringing the active agent into association with a carrier which constitutes one or more accessory ingredients. Compositions containing agents according to the invention may be administered as aerosols and inhaled. Compositions suitable for parenteral administration conveniently comprise a sterile aqueous or non-aqueous preparation of agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butanediol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

### Targeting Agent

It may be desirable to modify the carrier according to the invention to target a carrier complex to a cell type or organ to increase efficacy and reduce side effects. Targeting means are known in the art and include antibodies to cell surface receptors and ligands that bind cell surface receptors. Also included are ligands that bind intracellular targets to facilitate cell uptake of the carrier complex. In some instances the targeting agent and therapeutic agent is the same agent. For example, the over-expression of cell growth factors by cancer cells, for example VEGF receptors, can be targeted using antagonistic antibodies crosslinked to the carrier thereby homing the complex to cells expressing the receptor. Further examples include tumour rejection antigens which are uniquely expressed by cancer cells. Tumour rejection antigens are well known in the art and include, by example and not by way of limitation, the MAGE, BAGE, GAGE and DAGE families of tumour rejection antigens, see Schulz et al PNAS, 1991, 88, pp 991-993.

### Imaging Agent

An “imaging agent” is an agent capable of detection, for example by spectrophotometry, flow cytometry, or microscopy. For example, a label can be attached to the carrier, thereby permitting detection of the carrier in vivo. Examples of imaging agents include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Fluorophores are imaging agents commonly used in the art. A fluorophore is a chemical compound, which when excited by exposure to a particular stimulus, such as a defined wavelength of light, emits light (fluoresces), for example at a different wavelength (such as a longer wavelength of light). Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) eliminates the need for an external source of electromagnetic radiation, such as a laser.

### Linking Agents

Cleavable chemical linking agents are known in the art and include agents that are reactive with carbohydrate binding moieties or with the biologically active agent according to the invention. The link between oligomeric complex and agent can be non-covalent [e.g., via van der Waals forces or hydrophobic interactions] or covalent via cleavable chemical linkers. In either respect the linked complex maintains a physical and functional association between the carrier and the biologically active agent such that the activity of the agent is not inhibited while associated with the carrier and the agent is readily cleaved from the carrier. The linking agent similarly is biodegradable and optionally metabolized by cells/organs after administration.

Epichlorohydrine or other epoxi containing cross linkers—e.g. similar to glycidols: All crosslinkers similar to epichlorohydrine or to glycidol that react with hydroxyl groups of the carbohydrate monomers. Epichlorohydrine forms ether bonds with carbohydrates in alkaline solution. Two carbohydrates appear to be linked by a glycerol moiety upon cross linking with Epichlorohydrin. Hydrolysis of the two epichlorohydrin cross linked carbohydrate moieties under physiological condition liberates the carbohydrate monomers and glycerol. It will be apparent to the skilled artisan that any component that has a similar chemistry to epichlorohydrin could work.

### Amino Acids:

### Glutamic Acid

Glutamic acid is a bifunctional carboxylic acid that might undergo esterification reactions with carbohydrates. The esterification might be mediated by activating agents or by heat under acidic conditions. Other crosslinking chemistry is potentially possible with sugars e.g. formation of carbonate like linkages, carbonic acid esters etc.

Lysine:

L-Lysine has to amino functionalities that might directly react with aldehyde groups of carbohydrates to form shiff's bases. Alternatively the amino groups might react with activated hydroxyl groups of the carbohydrates to form carbamate like linkages.

### In General Amino Acids:

e.g. Alanine

Alanine would be especially advantageous due to its ability to enter fast and efficiently into the energy cycle. Various combination of the above mentioned crosslinking chemistries are possible. e.g. first step—esterification of alanin with carbohydrates, second step activation of hydroxy groups in Alanine-carbohydrate conjugate to facilitate reaction with amino group in the Alanine-carbohydrate conjugate.

### The Drug Itself:

e.g. a drug that has two or more carboxylic acid groups (such as 2,4-pyridinedicarboxylic acid) that can be activated and subsequently react with carbohydrates to form a polymer. This has the advantage of very high loading efficiency e.g. 50% loading efficiency or more.

### Cancer Cells

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

### Stem Cells

The term “stem cell” represents a generic group of undifferentiated cells that possess the capacity for self-renewal while retaining varying potentials to form differentiated cells and tissues. Stem cells can be pluripotent or multipotent. A pluripotent stem cell is a cell that has the ability to form all tissues found in an intact organism although the pluripotent stem cell cannot form an intact organism. Furthermore, it is known that human somatic cells can be re-programmed to an undifferentiated state similar to an embryonic stem cell. For example, WO2007/069666 describes re-programming of differentiated cells (e.g. mouse fibroblast cells) without the need to use embryonic stem cells. Nuclear re-programming is achieved by transfection of retroviral vectors into somatic cells that encode nuclear re-programming factors, for example Oct family, Sox family, Klf family and Myc family of transcription factors. The somatic cells de-differentiate and express markers of human embryonic stem cells to produce an “induced pluripotent cell” [iPS]. In Takahashi et al [Cell vol 131, p861-872, 2007] adult human dermal fibroblasts with the four transcription factors: Oct3/4, Sox2, Klf4, and c-Myc de-differentiate to human ES cells in morphology, proliferation, surface antigens, gene expression, epigenetic status of pluripotent cell-specific genes and telomerase activity.

A multipotent cell has a restricted ability to form differentiated cells and tissues. Typically, adult stem cells are multipotent stem cells and are the precursor stem cells or lineage restricted stem cells that have the ability to form some cells or tissues and replenish senescing or damaged cells/tissues. Generally they cannot form all tissues found in an organism, although some reports have claimed a greater potential for such ‘adult’ stem cells than originally thought. Examples of multipotent stem cells include mesenchymal stem cells. Mesenchymal stem cells differentiate into a variety of cell types that include osteoblasts, chondrocytes, myocytes, adipocytes and neurones. Typically, mesenchymal stem cells are obtained from bone marrow. Currently, stem cell therapies are exploring different sources of pluripotent and multipotent stem cells and cell culture conditions to efficiently differentiate stem cells into cells and tissues suitable for use in tissue repair.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

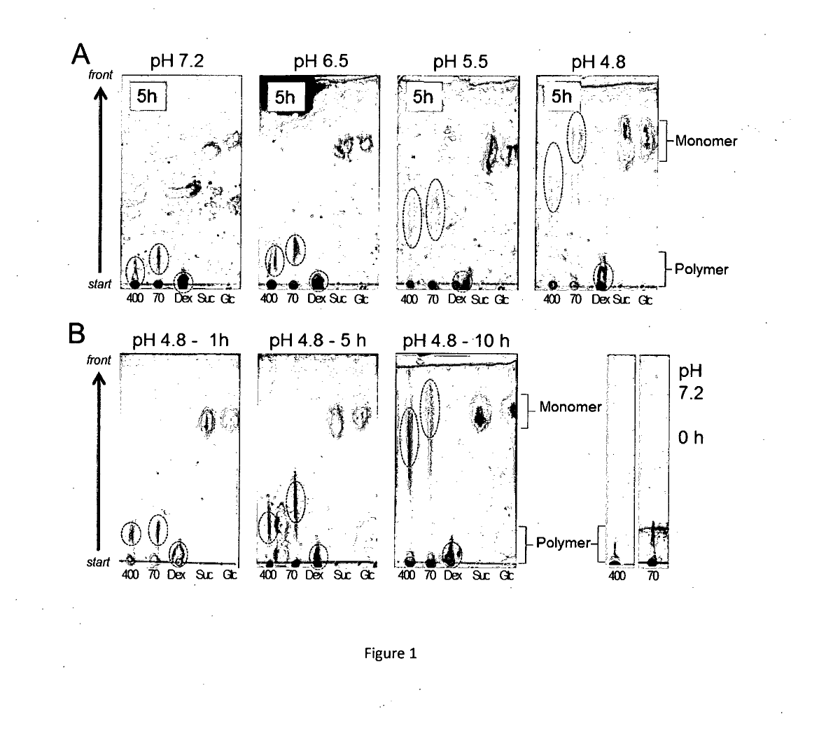

FIG. 1 illustrates that Ficoll depolymerises to monosaccharides in vitro at lysosomal pH as evidenced by thin layer chromatography. A) Decay characteristics of Ficoll 400 (400), Ficoll 70 (70), dextran 670 kDa (Dex), and standards for sucrose (Suc) and Glucose (Glc), which had been exposed for 5 hrs to buffer solution imitating extracellular space (pH 7.2), endosomes (6.5), late endosomes (pH 5.5) and lysosomes (pH 4.5). Ficolls disintegrate faster into smaller subunits towards the lysosomal pH. B) To emulate a time course within lysosomes, Ficolls and dextran were dissolved in a buffer solution with a pH of 4.8. Aliquots of these solutions were analysed after 1, 5 and 10 hours by thin layer chromatography to monitor fragmentation of polymer. Control observation was done at pH 7.2. Sizes of Ficoll degradation fragments begin to reach that of monomeric Glc or Suc after 10 hrs. Dextran remained stable under these conditions;

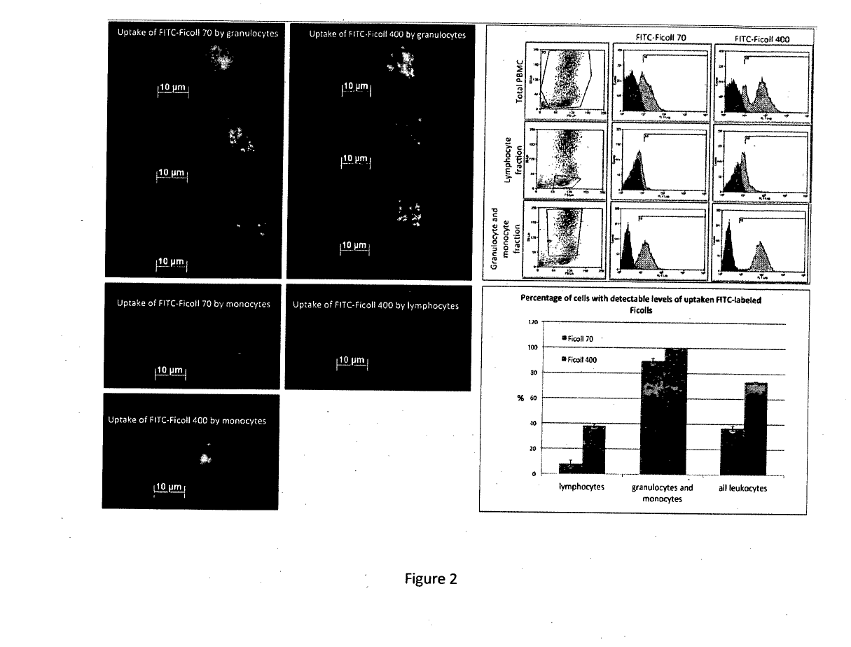

FIG. 2: illustrates that uptake within 30 minutes of TRITC-tagged Fc70 and Fc400 into mononuclear cells during preparation of buffy coats. Buffy coats were prepared from peripheral blood using Ficoll-Paque and a standard protocol. During contact and preparation (centrifugation) time, 1 μM of each fluorochrome-tagged polymer was present. After extensive washing, buffy coats were analysed by flow cytometry, which revealed significant uptake into granular compartments of all cell types analysed;

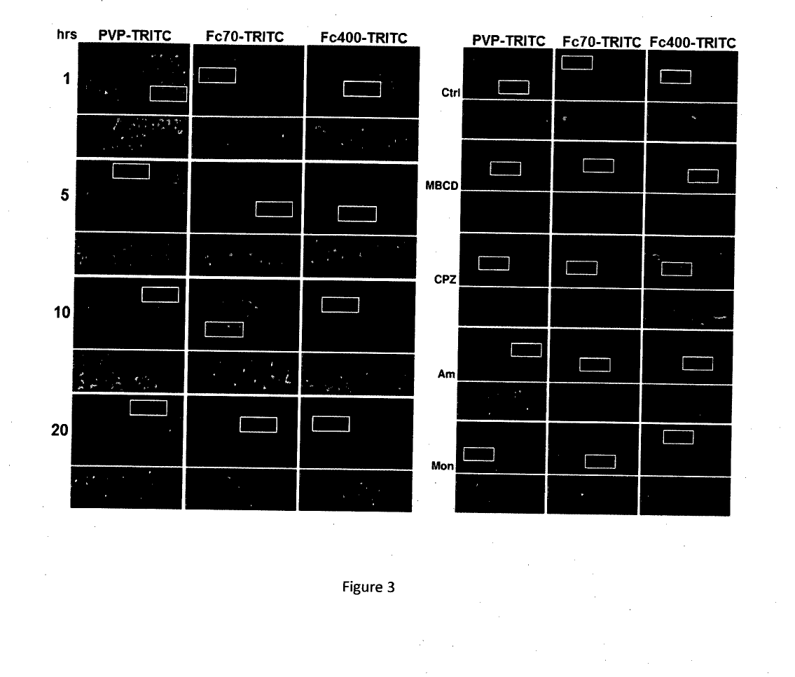

FIG. 3: illustrates that uptake of TRITC-tagged PVP, Fc70, and Fc400 into human mesenchymal stem cells and modulation of its uptake by pinocytosis inhibitors. A) Cells were pulsed for 1 hour with 1 μM of each polymer and monitored for 20 hrs. PVP showed a persistent granular/endosomal pattern with late peripheral distribution. In contrast, Fc70 and Fc400, accumulated in worm-like compartments after 1 hour, then occurred in a granular/endosomal pattern and, finally with a peripheral distribution at 20 hrs. B) Cells exposed for 1 hour to pinocytosis inhibitors methyl-beta-cyclodextrin (MβCD) (10 mM), chlorpromazine (CPZ) (28 μM), amiloride (Am) (300 μM), and monensin (Mon) (10 μM) were incubated for a further 0.1 hour with a mixture of each inhibitor and each TRITC-labelled polymer. MβCD completely inhibited uptake of all polymers, while Am showed no significant effects. CPZ reduced polymer presence across the board, but trapped Fc in the worm-like compartments. Monensin did not inhibit uptake of polymers but arrested them in a seam of vesicles at the periphery of the cells;

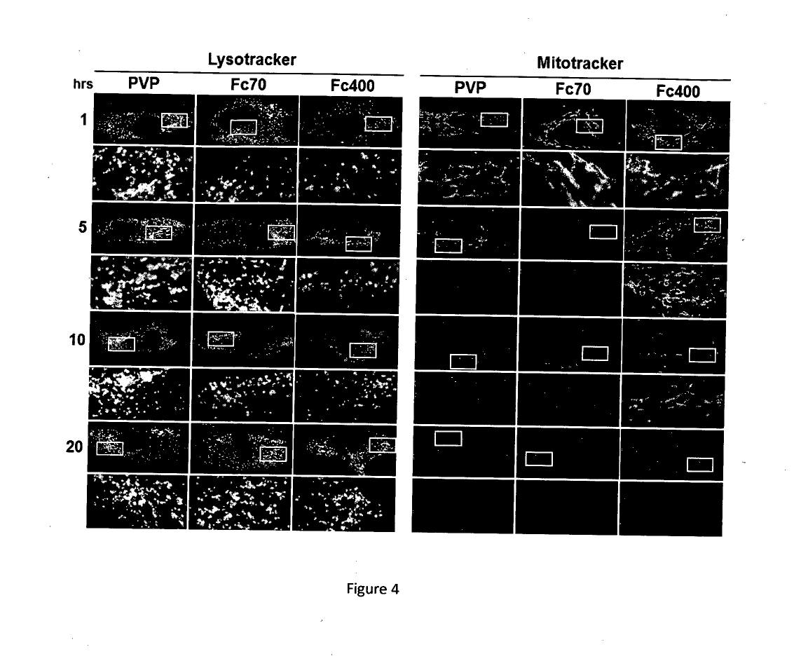

FIG. 4: illustrates the tracking of intracellular routing of TRITC-tagged PVP, Fc70, and Fc400 in human mesenchymal stem cells A) Lysosomal Tracking: cells were incubated with polymer (red fluorescence) for 1 hour, and for a further 15 mins with Lysotracker® (green fluorescence). Superimposition of images and resulting yellow mix colour indicate colocalisation. PVP is taken up immediately into a subset of lysosomes and stays in this location for the duration of the experiment. Both Ficolls, particularly Ficoll 400, show an immediate disparate localisation in worm-like organelles, with a small portion of lysosomes labelled, and then a transition of pattern towards lysosomal compartment after 20 hrs. B) Mitochondrial tracking: Cells were incubated with polymer for 1 hour, and for a further 15 mins with Mitotracker® (green fluorescence). PVP is taken up immediately into granules, with traces also in the mitochondrial compartment, while both Ficolls show a clear regional colocalisation with mitochondria after 1 hour. At later time points both Ficolls are redistributed to a peripheral granular compartment. Ficoll 400 showed a particularly good colocalisation with mitochondria, which persists up to 5 hrs;

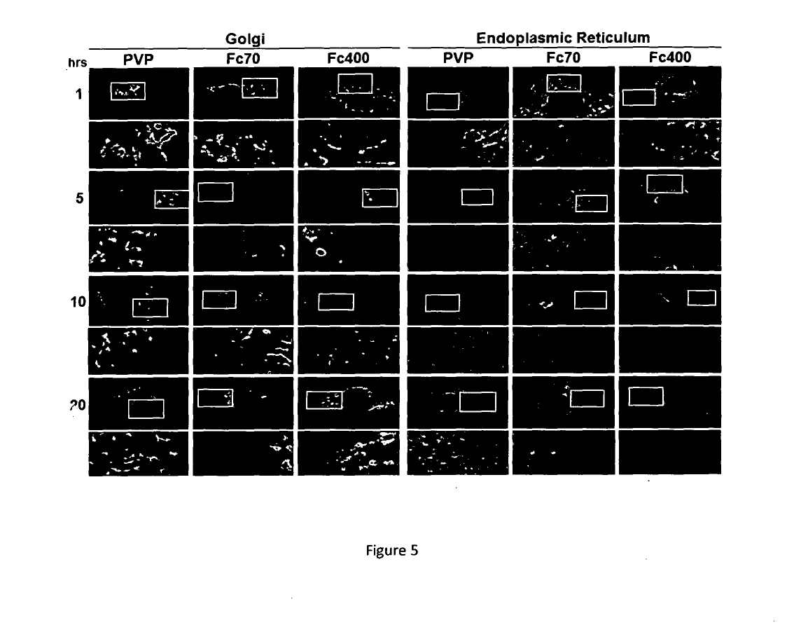

FIG. 5: illustrates the micropinocytosed TRITC-tagged PVP, Fc70-TRITC and Fc400-TRITC into human mesenchymal stem cells do not enter the Golgi apparatus or the endoplasmic reticulum. Cells were incubated with each TRITC-labelled polymer for 1 hour, and for a further 15 mins with ER tracker and NBD C₆-ceramide to selectively visualise the endoplasmic reticulum and Golgi apparatus, respectively. Superimposition of images and resulting yellow mix colour would indicate colocalisation. All polymers did not show significant colocalisation with ER. The Ficolls did not show significant colocalisation with the Golgi apparatus, but traces of PVP appeared to be present in the Golgi apparatus after 1 hour, which were absent after 5 hours;

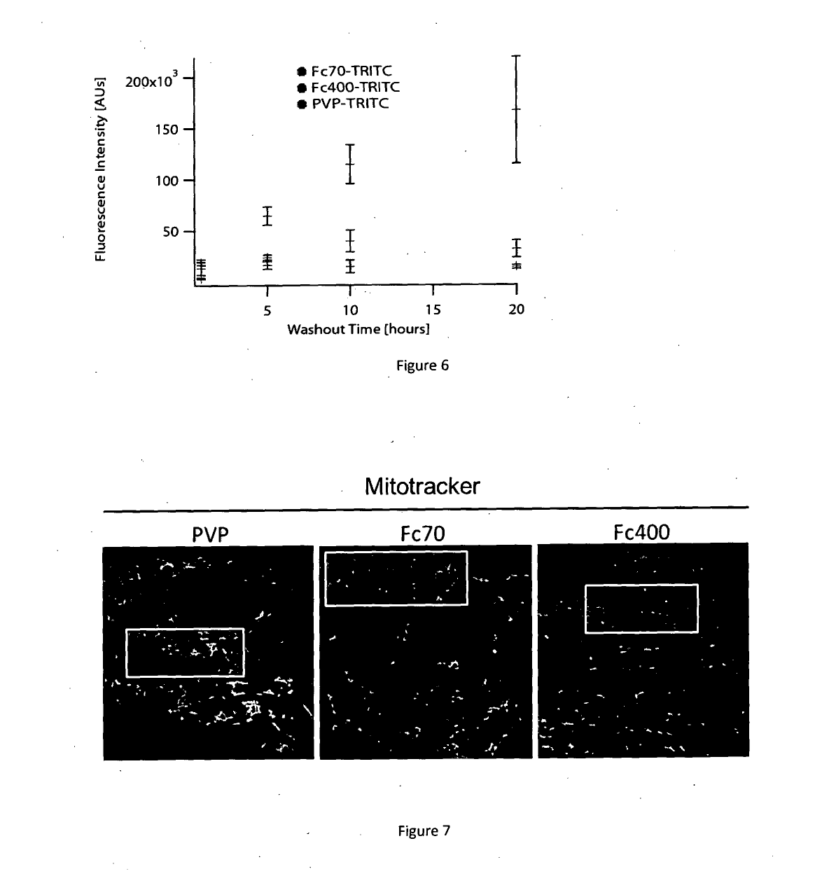

FIG. 6: illustrates the release of pinocytosed fluorochrome from human mesenchymal stem cells is smallest with PVP. Cells were pulsed for 1 hour with 1 μM of TRITC-tagged PVP, Fc70 and Fc400, respectively, washed and release of fluorescence into culture medium was monitored via fluorescent spectrometry from 5 to 20 hrs. PVP or fluorochrome-tagged fragments of it were hardly detected after 20 hours, while Fc400 associated fluorescence was continuously released up to 20 hours, while Fc70 reached a release peak at 10 hours. This suggests intracellular retention of PVP without significant degradation, while Ficolls are increasingly degraded;

FIG. 7: illustrates tracking the mitochondrial routing of TRITC-tagged PVP360, Ficoll 70 and Ficoll 400 in human Wi-38 embryonic lung fibroblasts. Cells were incubated with each polymer (red fluorescence) for 1 hour and for a further 15 minutes with Mitotracker® (green fluorescence). Superimposition of images and the resulting mixed yellow colour indicate colocalisation. In this cell type, all three polymers colocalised with mitochondria after 1 hour;

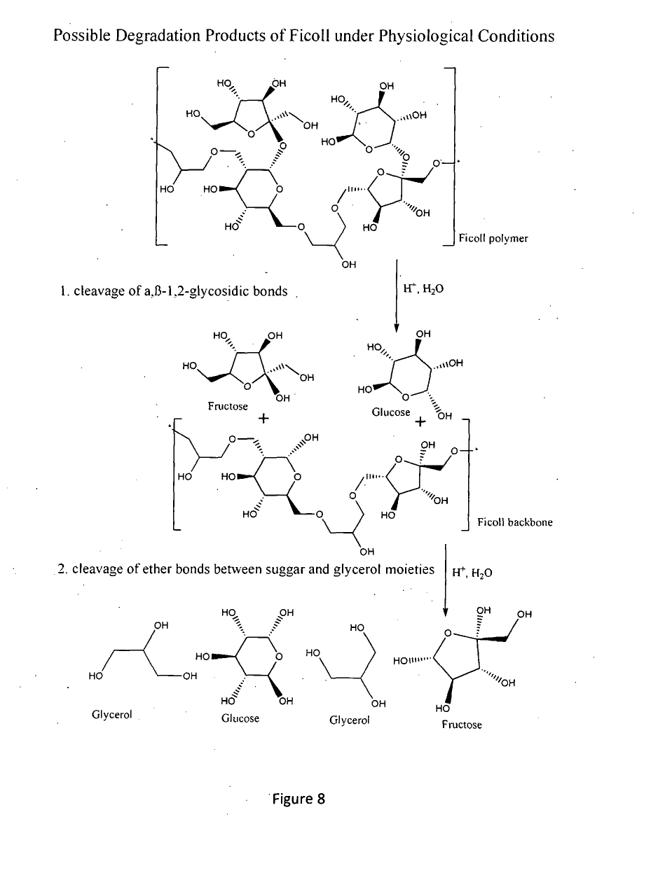

FIG. 8: illustrates a schematic drawing of possible in vivo degradation of the polysucrose polymer Ficoll. The possible degradation products of Ficoll are glucose, fructose and glycerol. All of these breakdown products are non-toxic and are known to be essential to the survival of mammalian cells;

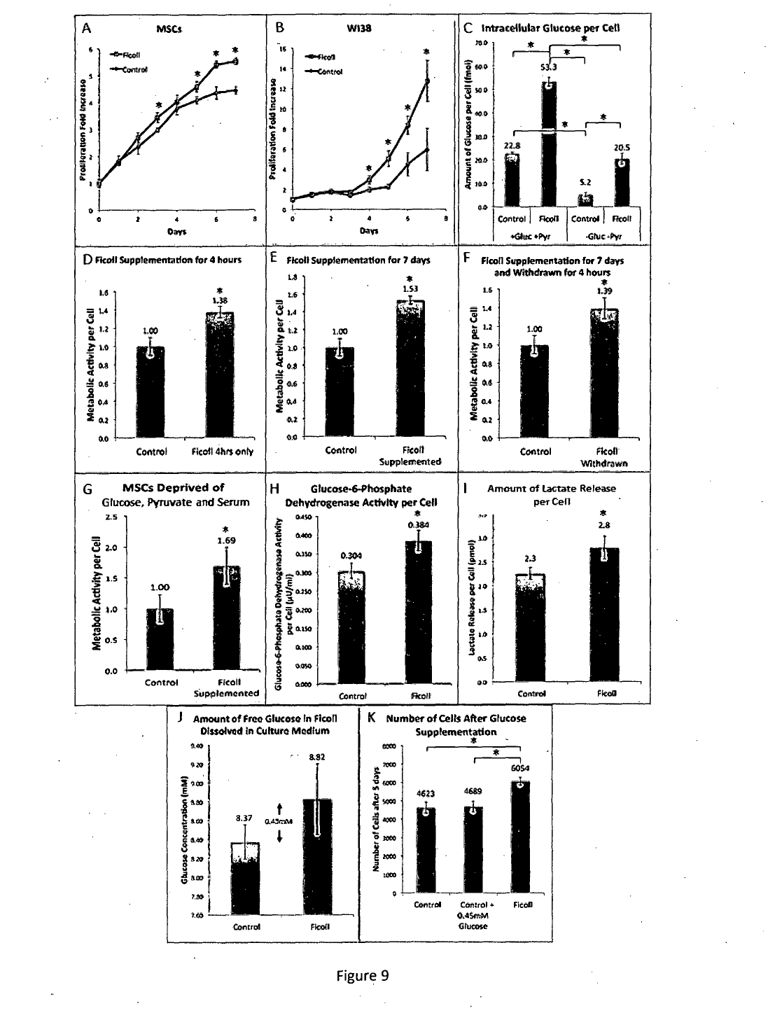

FIG. 9: illustrates that Ficoll supplementation increases proliferation, increases metabolic activity and substitutes for glucose and pyruvate depletion. (A, B) Addition of Ficoll mix to standard cultures increases proliferation of hMSCs by 25% and of fibroblasts by 100%; (C) hMSCs cultured with Ficoll show increased intracellular glucose content, while Ficoll supplementation of glucose and pyruvate starved hMSCs rescues intracellular glucose content to levels similar to hMSCs in standard low glucose medium; (D) hMSCs cultured with Ficoll for only 4 hours prior to MTS assay showed increased metabolic activity as well as (E) hMSCs cultured with Ficoll for 7 days and (F) hMSCs cultured with Ficoll for 7 days and without Ficoll 4 hours immediately before measurement; (G) Ficoll supplementation increases the metabolic activity of hMSCs deprived of glucose, pyruvate and serum; (H) Glucose-6-phosphate dehydrogenase activity is increased by Ficoll supplementation; (I) hMSCs supplemented with Ficoll showed increased lactate production; (J) 0.45 mM of glucose was detected when Ficoll is dissolved in culture medium; (K) hMSCs dosed with 0.45 mM of glucose did not show significant increase in cell numbers *p value <0.05 by Student's T Test;

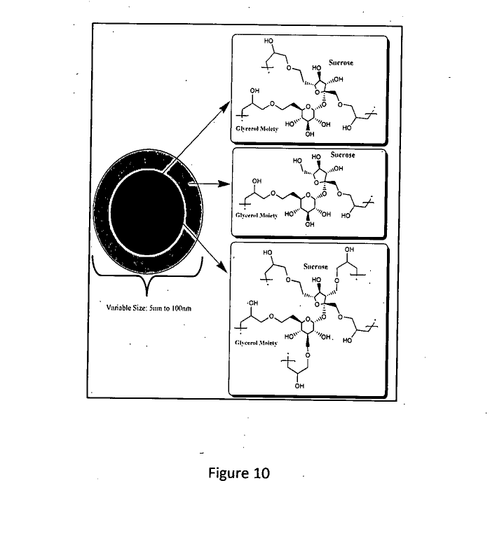

FIG. 10: illustrates the synthesis of metabolizable nanoparticles of different sizes. Schematic drawing about the possibility to engineer degradation profiles for carbohydrate based macromolecules based on crosslinking density. Depending on the nature of the specific drug that needs to be delivered to a target tissue, the size of the metabolizable nanoparticle (mNP) can be varied between 5 and 100 nm. The degradation profile might be engineered by stepwise growing of the nanoparticles with different cross linking densities. The use of epichlorhydrin as a chemical crosslinker leads to a structure in which sugar monomers are crosslinked by glycerol moieties. In this figure, we have depicted crosslinking between sucrose subunits. [The structure of Ficoll consists of sucrose subunits crosslinked by epichlorhydrin]. Using epichlorhydrin as a crosslinker allows us to determine degradation profiles of mNPs, control their size and shape, combine smaller units of mNPs into larger agglomerates, and incorporate drug;

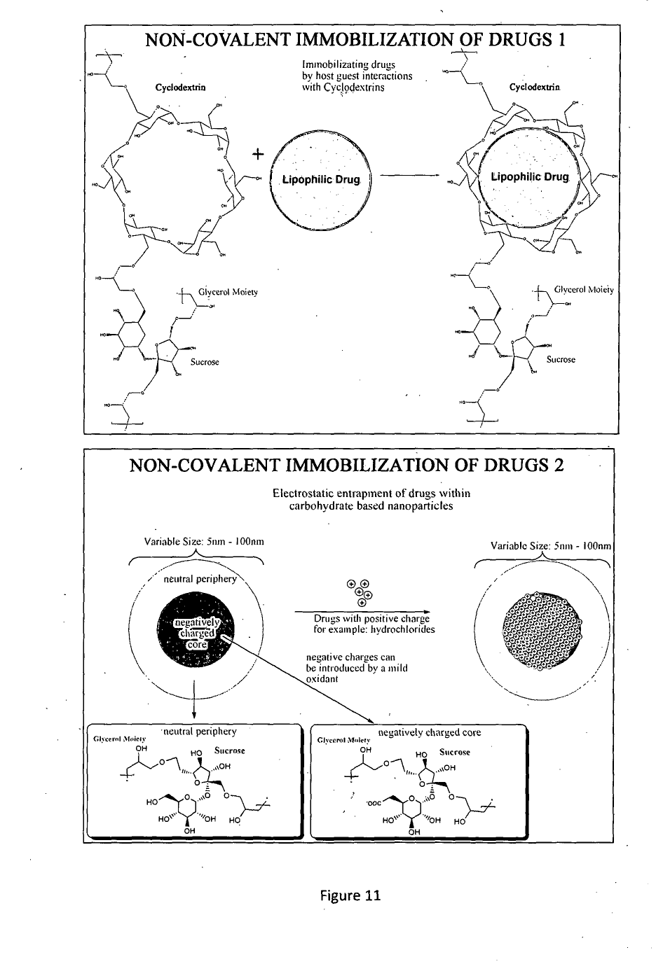

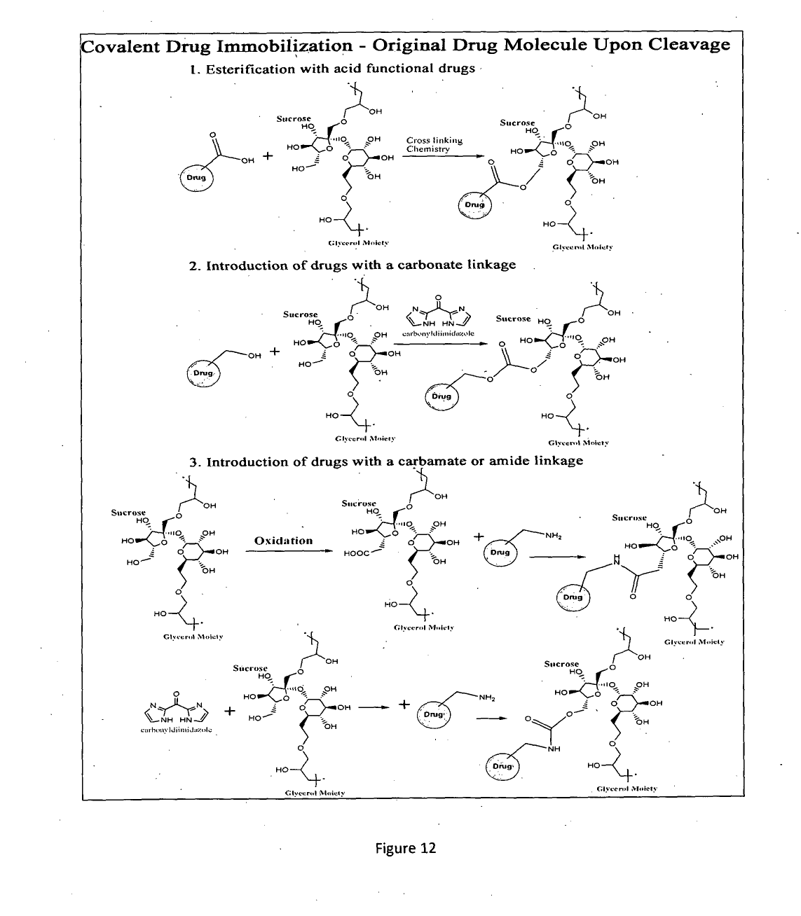

FIG. 11 (Top) Schematic representation of the synthetic pathway to immobilize lipophilic drugs in mNPs by incorporating cyclodextrins in the polymer backbone. Cyclodextrins are reactive to epichlorhydrin in the same way as sucrose or glucose, they can thus easily be incorporated into the polymers backbone where they entrap lipophilic drugs. (Bottom)) Schematic drawing of a possibility to entrap drugs (e.g. amines) non-covalently by electrostatic interactions. Oxidizing parts of the carbohydrate based polymer might introduce carboxylic acid groups which are negatively charged upon dissociation of the respective salt; and

FIG. 12 illustrates the envisioned possibility to conjugate drugs covalently carbohydrate based macromolecules. Various chemical approaches can be chosen to conjugate drugs to carbohydrate based macromolecules. (1) Drugs with acid functionality can be activated and directly bound to the macromolecule, hydrolysis yields the active drug. (2) Drugs with alcohol functionality can be activated for example with carbonyldiimidazole and bound to the carbohydrate based macromolecule via a carbonate linkage, hydrolysis might yield the initial drug. Amine functional drugs can be linked to carbohydrate based polymers by amide linkages when the carbohydrate based polymer was previously oxidized hydrolysis leads to the initial drug. (3) Amine functional drugs might also be conjugated by activating the carbohydrate based polymer with CDI which leads to a carbamate linkage, which releases the drug in its initial state upon hydrolysis.

### MATERIALS AND METHODS

### Macromolecular Crowders and Fluorescent Polymer Preparation

Ficoll 70 and Ficoll 400 conjugated to tetramethylrhodamineisothiocyanate (TRITC) were purchased from TdB Consultancy AB (Uppsala, Sweden). Polyvinylpyrrolidione (PVP) was conjugated to TRITC via nitrene chemistry. The protocol involves the following steps: (1) TRITC dye was functionalised with ethylene diamine to introduce a free amine group; (2) PVP was treated with a photocrosslinker called 5-azido-2-nitrobenzoic acid N-hydroxysuccinimide ester and exposed to ultraviolet light for up to 1 minute; (3) amine-functionalised TRITC dye was mixed with the PVP-photocrosslinker conjugate in an equimolar ratio; (4) TRITC-conjugated PVP360 was purified using a spin-column (MWCO=100,000 kDa). Each fluorophore-labelled polymer was dissolved in Hanks Buffered Salt Solution (HBSS). For all cell labelling experiments, a concentration of 1 μM of each polymer was used. Unlabelled Ficoll 70 and 400 were purchased from GE Healthcare (Uppsala, Sweden) and used as a mixture (37.5 mg/ml Ficoll 70 and 25 mg/ml Ficoll 400, the“Ficoll mixture”).

### Cell Culture

Human mesenchymal stem cells (hMSCs, Lonza) were seeded on 8-well Lab-Tek chamber slides with a borosilicate bottom (NUNC), at 10,000 cells per well in low glucose Dulbecco's Modified Eagle Medium (LGDMEM, 5.6 mM glucose, Gibco-Life Technologies) with 10% fetal bovine serum (FBS, Gibco-Life Technologies) and penicillin-streptomycin. Cells were incubated at 37° C. in 5% CO2. After 16 h, cells were separately incubated with TRITC-tagged polymers for 1 h in serum-free and antibiotic-free medium and then thoroughly washed 3 times with Hanks Balanced Salt Solution (HBSS). Then, phenol red-free and serum-free LGDMEM was added to the hMSCs in the Lab-Tek chamber slides. Cells were subsequently imaged with a confocal microscope.

### Confocal Imaging

Cells were viewed with a FV300 laser scanning confocal microscope (Olympus, Japan). The excitation beam from a 543-nm HeNe ion laser (MellesGriot, Singapore) was focused by a water immersion objective (60×, NA1.2, Olympus) into the fluorescent sample. For organelle co-localization studies, an additional 488-nm Ar laser (MellesGriot, Singapore) was used to excite fluorescent organelle labels. Images were acquired with the Olympus FV300 software.

### Polymer Washout Experiments

hMSCs were incubated with each TRITC-tagged polymer for 1 hour, then were washed 4 times with phenol- and serum-free LGDMEM. The supernatant from the 4th wash was retained and set aside as a baseline measurement at the 1-hour time point. For the 5-, 10- and 20-hour time points, cells were incubated in phenol red-free and serum-free LGDMEM and the supernatant removed for measurement at the corresponding time points. The fluorescence intensity of each sample was measured using a PheraStar fluorimeter (BMG Instruments, Offenburg, Germany). All measurements were performed in triplicate.

### Pinocytosis Inhibition Studies

hMSCs (Lonza) were pre-incubated for 1 hour with 10 mM methyl beta cyclodextrin (MBCD, Sigma-Aldrich), 28 μM chlorpromazine (CPZ, Sigma-Aldrich), 300 μM amiloride (Am, Sigma-Aldrich), and 10 μM monensin (Mon, Sigma-Aldrich) in serum-free LGDMEM. The cells were then incubated for a further 1 hour with a mixture of each inhibitor and each TRITC-labelled polymer in serum-free LGDMEM.

### Organelle Co-Localization Studies

hMSCs were incubated with each TRITC-labelled polymer for 1 hour in phenol red-free and serum-free LGDMEM and were then co-labelled with the following fluorescent organelle labels for a further 15 mins: Lysotracker™ (50 nM) for lysosomes, Mitotracker™ (100 nM) for mitochondria, ER Tracker™ (1 μM) for the endoplasmic reticulum and NBD C6-ceramide (1 μM) for the Golgi apparatus. All organelle labels were purchased from Invitrogen (Singapore).

### Peripheral Blood Mononuclear Cell (PBMC) Studies

Peripheral blood was obtained from the National University Hospital blood bank or from healthy donors. Peripheral blood mononuclear cells (PBMCs) were isolated via gradient centrifugation over Ficoll-Paque (Sigma) following the manufacturer's instructions. Blood was diluted with the same amount of PBS containing 2M EDTA. 22 ml of diluted blood was layered over 14 ml of Ficoll-Paque and centrifuged at 400 g for 30 min. A buffy coat ring was collected from separated blood samples and washed twice with PBS containing 2 mM EDTA. PBMCs were then seeded in phenol red-free LGDMEM containing either TRITC-tagged Ficoll 70 or Ficoll 400on non-adherent dishes for 1 hour. Afterwards cells were collected and fixed in 1% formaldehyde for 15 min. Fixed cells were analyzed either using the Cyan flow cytometer (DakoCytomation) or resuspended in PBS buffer supplemented with 0.5% FBS for further staining. Cell nuclei were stained with DAPI and the cytoskeleton with Alexa Fluor 594-labelled Phalloidin for 30 min. Cells were washed once with PBS buffer supplemented with 0.5% FBS and re-suspended in PBS. Cells were then distributed between two coverslips and viewed with a Zeiss apoptome fluorescence microscope.

### Hydrolytic Decay Studies

Thin layer chromatography (TLC) of carbohydrates was performed with silica gel on aluminium support plates (5×7.5 cm, Schleicher & Schuel GmbH, purchased through Sigma-Aldrich Singapore). Ficoll 70, Ficoll 400, dextran 670 kDa, glucose and sucrose were incubated in 1× PBSbuffer at 37° C. at different pH representing the extracellular space (pH 7.2), early endosome (Ph6.5), late endosome/secretory vesicle (pH 5.5) and lysosome (pH 4.8). Loaded TLC plates were placed in a beaker with 0.5 cm solvent level (Ethylacetate:Methanol:Water; 52:36:13) and plates were taken out shortly before the solvent front reached the end of the plate. Plates were air-dried, immersed briefly in 5% w/w sulfuric acid and briefly placed on a hotplate at 150-200° C. Developed plates were scanned with a commercial photo scanner. Sample and mobile phase positions were marked and the coefficient of refraction was determined by dividing the total length of the solvent front through the migration length. Further assessment of the degradation process was achieved by not only comparing the refraction coefficients but also the distribution of degradation products for each single sample around the refractive index. Cell culture of MSCs and fibroblasts for proliferative and metabolic assays hMSCs and normal fetal lung fibroblasts (WI38; American Tissue Culture Collection) were routinely cultured in LGDMEM and High Glucose Dulbecco's modified Eagle's medium (HGDMEM, Gibco-Life Technologies), respectively, with 10% fetal bovine serum (Gibco-LifeTechnologies) at 37° C. in a humidified atmosphere of 5% CO2.

### Proliferation Assay Under Macromolecular Crowding

hMSCs and WI-38 fibroblasts with an initial seeding density of 3000 cells/cm2 and at 2000 cells/cm2, respectively, were monitored daily for absolute cell numbers. Every day a replicate culture plate with cells grown under standard and mixed macromolecular crowding conditions was randomly selected and cells were fixed in absolute methanol at −20° C. and stained with nuclear dye 4′,6-diamidino-2-phenylindoldilactate (DAPI). Adherent cytometry was done by acquiring nine image sites, covering 71% of the total well area, at 2× magnification using a Nikon TE600 fluorescence microscope plus Xenon illuminator (LB-LS/30, Sutter InstrumentCompany, Novato, Calif., USA) with an automated Ludi stage (Bioprecision 2, Ludl ElectronicProducts Ltd, Hawthorne, N.Y., USA) and analyzed using Metamorph® Imaging System Software (Molecular Devices, Downingtown, Pa., USA) to acquire the number of nuclei per well. The initial number of cells was calculated from nuclei counts from the replicate plate fixed 24 hours after seeding. The final number of cells on each subsequent day was calculated from replicate plates fixed on the respective days. The increase in proliferation was given by the ratio of final number to initial number of cells.

### Intracellular Glucose Assay and Free Glucose Dosing Effect on Proliferation

In order to monitor intracellular glucose generation from Ficoll, MSCs were cultured for 3 days under standard conditions or in the presence of Ficoll mixture. In addition cells were deprived of glucose and pyruvate, but supplemented in parallel experiments with the Ficoll mixture. Cell lysates were generated in CHAPS buffer and centrifuged at 12,000 g for 20 mins at 4° C. Supernatants were analyzed using the Glucose and Sucrose Assay Kit (Abcam) according to the manufacturer's instructions, and quantification of glucose oxidase reaction product resorufin was performed by colorimetric absorbance plate readings using an Infinite 200 absorbance plate reader (Tecan) and analyzed by Tecan i-Control software. The same kit was used to assess any carry-over of free glucose after dissolving Ficoll in standard culture medium (LGDMEM/10% FBS in LGDMEM). The resulting value—0.45 mM—was added as monomeric glucose to standard culture medium for hMSCs to ascertain its effects on proliferation after 5 days by adherent cytometry (see above).

### MTS Assay and Glucose, Pyruvate and Serum Deprivation

hMSCs were seeded at 3400 cells/well in 24 well plates (CelStar, Greiner Bio-One) and cultured for 7 days under standard conditions in standard culture medium with or without the Ficoll mixture. The metabolic activity in each well was then measured using the CellTiter 96® AQueous One Solution Cell Proliferation Assay (Promega) according to the manufacturer's instructions. The MTS tetrazolium compound in the assay is bioreduced by cells, using NADPH or NADH produced in metabolic reactions, into a colored formazan product. The change in absorbance was measured by Infinite 200 absorbance plate reader (Tecan) and analyzed by Tecan i-Controlsoftware. The plates were then fixed with methanol, stained with DAPI and the number of cells calculated by adherent cytometry. The metabolic activity from each well was normalised to the respective number of cells and further normalised to the respective controls. hMSCs were seeded at 3400 cells/well in standard culture medium in a 24 well plate for 24 hours and the medium was changed LGDMEM devoid of glucose and pyruvate. Test cultures received the Ficoll mixture. After 7 days of culture, the metabolic activities were measured. The metabolic activity from each well was normalised to the respective number of cells and further normalised to the respective controls using DAPI staining as above.

Glucose-6-phosphate Dehydrogenase Assay and Lactate Release Assay

hMSCs were cultured for 3 days under standard culture conditions with or without the Ficoll mixture. The culture medium was collected and centrifuged at 12,000 g for 20 mins at 4° C. The lactate in the supernatant was measured using the Lactate Colorimetric Assay (Abcam) according to the manufacturer's instructions. The remaining cell layers were then lysed with CHAPS buffer and incubated on ice for 30 mins. The lysate was then centrifuged at 12,000 g for 20 mins at 4° C. and the glucose-6-phosphate activity in the supernatant was measured using the G6PD Colorimetric Assay (Abcam) according to the manufacturer's instructions.

### Example 1

Typically, crosslinking reactions are adapted to needs and scale which would be within the skill of the artisan and would not require extensive experimentation.

### Epichlorohydrine

The carbohydrate and the crosslinker epichlorohydrin is mixed in aqueous alkaline solution in a desire molar ratio. The molar ratio is at least 1:1 and maximally limited by the number of reactive groups (towards epichlorohydrine) on the carbohydrate. Spontaneous cross linking occurs.

The cross linking density and size distribution can be controlled by the viscosity of the solution (concentration of monomers and crosslinkers) and be the reaction temperature (besides the molar ratio of the reactants). The pH of the reaction mixture (pH10-14) can be used to create a preference of epichlorohydrine for specific hydroxyl groups on the carbohydrate backbone. The preparation is allowed to react for a sufficient amount of time in order for epichlorohydrin to be completely consumed in the cross linking or hydrolysis reaction. The resulting polymer can be purified by dialysis or by other ultrafiltration methods.

It is possible to add new carbohydrate monomers and epichlorohydrin cross linker in a different molar ratio to the polymers that were prepared in the first step. The average molecular weight of the polymers subsequently increases by crosslinking and preferably growth.

This step might be repeated several times. This leads to the possibility to create a nanoparticle (polymer molecule) that has a striated structure in which each layer has a different crosslinking density and thus a different degradation kinetic under physiological conditions.

### Example 2

A drug that is non reactive to epichlorohydrin is bound to the carbohydrate monomer first, for example, retinoic acid and glucose.

The carboxy group in retinoic acid is activated (e.g. by carbonylimidazole) and allowed to react with glucose in a appropriate solvent (e.g. DMSO). The reaction product is subsequently purified. The retinoic acid-glucose ester is then subjected to epichlorohydrin cross linking (as described in example 1) by which the unreactive drug is not affected (cross links only happen between the glucose moieties).

Optionally new carbohydrate monomers and epichlorohydrin might be added to the preparation once the reaction is completed. Polymer growth leads to a nanoparticle with a core that contains the drug and a shell that is free of the drug.

Optionally the carbohydrate-drug monomer and the crosslinker might be added in several steps to the preparation. It is possible to adjust the monomer cross linker ratio in each step. This would lead to preferably striated nano particle with layer of different cross linking density and thus different degradation and drug release profiles under physiological conditions.

### Example 3

The polymer is first formed by crosslinking of carbohydrates with epichlorohydrin and an activated drug is linked to the polymer subsequently.

### Amino Functional Drugs

The carbohydrate moieties might be oxidized e.g. by sodium meta periodate to create aldehyde groups in the polymer. The amino functional drug can then be bound by Schiff's base linkages. Schiff's bases also called (mines are subjected to a moderate hydrolysis under physiological conditions. The Imine can optionally be reduced to from an amine (with sodium borohydride or similar reducing agents) this would lead to a stronger bond that cannot be hydrolysed.

Carboxylic acid groups might be introduced into the polymer followed by subsequent activation of the carboxylic acid groups in order to form amide bonds with the drugs. Monochloracetic acid reacts with carbohydrates in aqueous alkaline solutions (pH 10 and greater) in a condensation reaction that introduces carboxylic acid groups to the polymer. Activation of the acid groups (e.g. to form a succiinimid ester) renders the polymer reactive towards amino groups.

### Carboxylic Acid Functional Drugs:

The acid functional drug might be activated e.g. by carbonyldiimidazole (CDI) or by other reagents that convert it to a highly reactive species towards hydroxyl groups. An appropriate solvent needs to be used. The carbohydrate-epichlorohydrin polymer tends to be soluble in DMSO which is suitable for CDI activation. The activated acid functional drug reacts spontaneously with hydroxyl groups to form ester linkages.

### Hydroxy Functional Drugs

The hydroxyl function might be activated e.g. with CDI. CDI activated hydroxyl groups react with other hydroxyl groups to form carbonic acid esters.

Thiol functional drugs: A thiol containing molecule e,g, the amino acid cysteine can be coupled to the polymer with the above mentioned chemistry via its carboxylic acid or amino group under reducing conditions. The thiol functional group can then form a disulfide bond with the cysteine modified polymer under oxidizing conditions.

The above described chemistry might also be used to couple the drug to a carbohydrate monomer followed by cross linking to a polymer (if the drug is non-reactive to the crosslinking agent)

### Example 4

The drug itself is the crosslinker. A drug that can act as a bifunctional crosslinker. Dicarboxylic acids such as 2,4-pyridinecarboxylic acid. The drug is activated with e.g. CDI in DMSO and mixed with the carbohydrate monomer e.g. glucose. Spontaneous polymerization occurs and might be accelerated by heating the preparation.

### Example 5

### Where Amino Acids are Used to Cross Link Carbohydrates

Di-amino functional amino acids such as 1-lysine. The amino acid might be subjected to esterification with a carbohydrate under acidic conditions in a appropriate solvent such as DMSO. The amino group is preserved due to salt formation under acidic conditions. The amino acid carbohydrate ester can be subjected to aqueous alkaline oxidizing conditions in which the formed aldehyde groups react with the free amine groups. Optionally additional carbohydrate monomers might be added in order to control the crosslinking density.

Di-carboxylic acid functional amino acids. The amino group needs to be protected e.g. by salt formation followed by activation of the acid functionalities e.g. by CDI. The activated acid groups react with carbohydrate monomers to form ester bonds. Optionally, the protected amino group might subsequently be activated and subjected to further cross linking.

### Example 6

### Binding of Macromolecular Drugs

Proteins, nucleic acid or other drugs might be bound by their native or artificially introduced functional groups with the same or similar crosslinking chemistry as described above.

Relatively low molecular weight oligomers or polymers typically smaller then 10 kDa might be formed by the above mentioned methods and equipped with specific and very selective moieties to facilitate cross linking. The moieties might be alkyne and azide moieties that form cross links by the so called click chemistry. The small molecular weight polymers with alkyne and azide functionalities are mixed with the macromolecular drugs in aqueous solution and subjected to a cue that induces cross linking. The macromolecular drugs (e.g. paclitaxel, poly amino acids, nucleic acids or others) are sterically encaged within the polymer and not affected by the crosslinking chemistry. The entrapment efficiency “mash size” might be controlled by the ratio of crosslinking moieties to monomers.

A more biodegradable alternative to click chemistry might be to use disulfide bonds. Oligomers smaller than 10 kDa are equipped with thiol groups e.g. by conjugating to cysteine. The oligomer-cysteine conjugates are mixed with certain macromolecular drugs under reducing conditions. The macromolecular drugs will be entrapped once the environment becomes oxidizing.

1. An oligomeric carrier complex comprising:
i) a carbohydrate based polymer comprising one or more monomeric or dimeric sugars;
ii) one or more biologically active molecules;
iii) a linkage agent that either directly or indirectly links or associates the carbohydrate polymer with the one or more biologically active molecules, wherein the polymer and linkage agent are biodegradable and the polymer and/or linkage agent is adapted to be metabolized by an organism or cell after administration; and optionally
iv) a targeting agent to target the conjugate to a cell or organ.

2. The complex according to claim 1, wherein said complex is a nanoparticle.

3. The complex according to claim 2, wherein said complex has a diameter of:
(i) 1 nm-1000 nm
(ii) 1 nm to 300 nm; or
(iii) 5 nm to 100 nm.

4.-5. (canceled)

6. The complex according to claim 1, wherein said monomeric sugar is glucose, galactose or fructose.

7. The complex according to claim 1, wherein said dimeric sugar is sucrose, lactose or maltose.

8. (canceled)

9. The complex according to claim 1, wherein said carbohydrate based polymer comprises one or more different monomeric or dimeric sugars.

10. The complex according to claim 1, wherein said biologically active molecule is a therapeutic agent.

11. The complex according to claim 10, wherein said therapeutic agent is a small organic molecule, a nucleic acid molecule, or a proteinaceous agent.

12. The complex according to claim 11, wherein said small organic molecule is a chemotherapeutic agent, antibiotic, or antiviral agent.

13.-15. (canceled)

16. The complex according to claim 11, wherein said proteinaceous agent is a therapeutic antibody, an active binding fragment of the therapeutic antibody, or a non-antibody pharmaceutical peptide or protein.

17. The complex according to claim 16, wherein said therapeutic antibody is a monoclonal antibody, a chimeric antibody, a humanized or human antibody.

18. The complex according to claim 16, wherein said active binding fragment is Fab, Fab₂, F(ab′)₂, Fv, Fc, Fd, or a single chain antibody fragment.

19.-20. (canceled)

21. The complex according to claim 11, wherein said nucleic acid molecule comprises an antisense RNA, an antisense oligonucleotide, a small interfering RNA (siRNA), a nucleic acid based vector, or a gene therapy vector adapted for expression.

22.-23. (canceled)

24. The complex according to claim 21, wherein said gene therapy vector is viral based.

25. The complex according to claim 1, wherein said targeting agent is an antibody or antibody fragment thereof.

26. The complex according to claim 25 wherein the antibody or antibody fragment is both the biologically active agent and the targeting agent.

27. The complex according to claim 1, wherein said targeting agent is a ligand for a receptor expressed by a target cell or organ and targeting of the oligomeric carrier complex is via ligand:receptor binding.

28. The complex according to claim 1, wherein said biologically active agent is associated with a second carrier moiety which is crosslinked or associated with the carrier.

29. The complex according to claim 28, wherein said second carrier is biodegradable.

30. The complex according to claim 29, wherein said second carrier is a dextrin.

31. The complex according to claim 1, wherein said biologically active agent is an imaging agent.

32. The complex according to claim 31, wherein the complex further comprises a therapeutic agent.

33. The complex according to claim 1, wherein said linking agent is cleavable and biodegradable.

34. The complex according to claim 33, wherein said linking agent forms a covalent linkage between the carbohydrate polymer and the one or more biologically active molecules.

35. The complex according to claim 33, wherein said linking agent forms a non-covalent linkage between the carbohydrate polymer and the one or more biologically active molecules.

36. A pharmaceutical composition comprising an effective amount of an oligomeric carrier complex according to claim 1 and a physiologically acceptable excipient.

37. (canceled)

38. A method of treating a subject having cancer, a disease or condition resulting from mitochondrial dysfunction, an acute wound, or a chronic wound, comprising:
administering an effective amount of the oligomeric carrier complex of claim 1 to the subject, thereby treating the cancer, disease or condition resulting from mitochondrial dysfunction, acute wound, or chronic wound.

39.-40. (canceled)

41. A method for delivery of one or more biologically active molecules to mitochondria; comprising:
contacting a cell with the oligomeric carrier complex of claim 1, thereby delivering the one or more biologically active molecules to mitochondria.

42. The complex according to claim 41, wherein the one or more biologically active molecules is a therapeutic agent, photo-sensitizing agent, imaging agent, nucleic acid based agent, or combinations thereof.

43.-49. (canceled)

50. An ex vivo method for the administration of an oligomeric carrier complex, comprising:
i) forming a preparation comprising an isolated cell sample obtained from a subject and the oligomeric carrier complex according to claim 1;
ii) incubating the cell preparation under conditions that allow uptake of the oligomeric carrier complex into one or more cell types contained in the sample; and optionally
iii) re-administering the cell preparation to said subject.

51.-55. (canceled)

56. A process for manufacturing a biodegradable oligomeric carrier complex, comprising:
i) forming a reaction mixture comprising a carbohydrate based carrier, a biologically active agent and optionally a cross-linking agent;
ii) incubating the reaction mixture under reaction conditions that cross-link the carbohydrate based carrier and the biologically active agent to form an oligomeric carrier complex; and optionally
iii) purifying the oligomeric carrier complex from the reaction mixture.

57.-59. (canceled)
